(19) United States
(12) Patent Application Publication
(10) Pub. No.: US 2024/0018480 A1
(43) Pub. Date: Jan. 18, 2024

(54) ENHANCEMENT OF EXTRACELLULAR VESICLE PRODUCTION BY LYSOSOME INHIBITOR

(71) Applicant: ExoTop Theragnostics Inc., Covina, CA (US)

(72) Inventor: Dongki KIM, Covina, CA (US)

(21) Appl. No.: 17/866,404

(22) Filed: Jul. 15, 2022

(51) Int. Cl. C12N 5/0775

(52) U.S. Cl. CPC C12N 5/0662; C12N 2501/999; C12N 2500/95

(57) ABSTRACT

Provided are methods and systems for an enriched production of high-quality extracellular vesicles (EVs) from a mammalian cell. In some cases, the methods may comprise culturing the cell in a chemically-defined protein-free (CDPF) medium with the addition of a lysosome inhibitor to increase production of EVs. In some cases, the CDPF medium is supplemented with additives.

### BACKGROUND

Extracellular vesicles (EVs) are a mediator of intracellular communications delivering selectively packaged bioactive molecules including miRNAs, proteins, lipids, and metabolites from donor to recipient cells. Although EVs have attracted much interest in diagnostic, therapeutic, and biological research, current EV production and isolation methods are not suitable for preparing high-quality EVs with minimized contaminants from living cells and in amounts needed for clinical applications. There is thus an unmet need for approaches that overcome inherent limitations of conventional methods for a scalable production method of high-quality EVs.

### SUMMARY

While the potential of EVs has widely explored for diagnostics, therapeutics, and biological research, there still remain challenges in enriched production and isolation of high-quality EVs that have high purity, low contaminants, and/or consistent size and characteristics. Since there may be a lack of consensus regarding the preparation and isolation steps of high-quality EVs, various methods to retrieve EVs from biofluids or cell-cultured media have been reported.

The present disclosure provides methods, systems, cells, and kits for an enriched production of high-quality EVs from various cell sources to address at least the abovementioned shortcomings noted for conventional EV production. In some embodiments, the cell comprises a mammalian cell that is cultured in a chemically-defined, protein-free (CDPF) medium and a lysosome inhibitor and has an upregulated expression of CD63. In some embodiments, the mammalian cell comprises a stem cell, an immune cell, a naïve cell, or an engineered cell. In some embodiments, the mammalian cell comprises a mesenchymal stem cell that is derived from a bone marrow, an umbilical cord, a placenta, or an adipose tissue. In some embodiments, the immune cell comprises a T cell and a NK cell.

The present application relates generally to the lysosome inhibitor that reduces internal acidification of the cell. In some embodiments, culturing the cell in the CDPF medium and the lysosome inhibitor results in upregulation of fatty acid gene expression. In some embodiments, the fatty acid gene comprises fatty acid synthase. In some embodiments, culturing the cell in the CDPF medium and the lysosome inhibitor decreases formation of lysosomes and/or autophagosomes by the cell.

In some embodiments, the lysosome inhibitor comprises a beclin-1 inhibitor. In some embodiments, the lysosome inhibitor comprises an inhibitor of autophagosome or autophagolysosome formation. In some embodiments, the lysosome inhibitor is selected from a group consisting of SP600125, U0126, 3-methyladenine, bafilomycin A-1, LY294002, SB202190, SB203580, and chloroquine. In some embodiments, the CDPF medium comprises hypoxanthine, thymidine, glutamine, glucose, essential amino acids, or vitamins, or a combination thereof.

In some embodiments, the enhanced production of EVs comprises an increased production of EVs and/or a decreased degradation of EVs by the cell cultured in the CDPF medium and the lysosome inhibitor than a cell cultured without culture in the CDPF medium and the lysosome inhibitor. In some embodiments, the culturing the cell in the CDPF medium and the lysosome inhibitor increases activities of EVs than without culturing the cell in the CDPF medium and the lysosome inhibitor. In some embodiments, the upregulated expression of CD63 comprises an increase in CD63 by the cell cultured in the CDPF medium and the lysosome inhibitor than a cell cultured without culture in the CDPF medium and the lysosome inhibitor.

In some embodiments, the EVs have a mean diameter of about 85 nm to about 236 nm. In some embodiments, the cell is cultured for between about 24 hours and about 72 hours. In some embodiments, the cell is cultured to about 70˜80% confluency.

Also provided herein is a system for enriched production of extracellular vesicles (EVs). In some embodiments, the system comprises a mammalian cell cultured in a chemically-defined, protein-free (CDPF) medium and a lysosome inhibitor and having an enriched expression of CD63. In some embodiments, the mammalian cell comprises a stem cell, an immune cell, a naïve cell, or an engineered cell. In some embodiments, the mammalian cell comprises a mesenchymal stem cell that is derived from a bone marrow, an umbilical cord, a placenta, or an adipose tissue. In some embodiments, the immune cell comprises a T cell and a NK cell. In some embodiments, the CDPF medium is supplemented with additives comprising hypoxanthine, thymidine, glutamine, glucose, essential amino acids, or vitamins, or a combination thereof. In some embodiments, culturing the cell in the CDPF medium and the lysosome inhibitor reduces internal acidification of the cell. In some embodiments, culturing the cell in the CDPF medium and the lysosome inhibitor results in upregulation of fatty acid gene expression. In some embodiments, the fatty acid gene comprises fatty acid synthase.

In some embodiments, culturing the cell in the CDPF medium and the lysosome inhibitor decreases formation of lysosomes and/or autophagosomes by the cell. In some embodiments, the lysosome inhibitor comprises a beclin-1 inhibitor. In some embodiments, the lysosome inhibitor comprises an inhibitor of autophagosome or autophagolysosome formation. In some embodiments, the lysosome inhibitor is selected from a group consisting of SP600125, U0126, 3-methyladenine, bafilomycin A-1, LY294002, SB202190, SB203580, and chloroquine. In some embodiments, the enhanced production of EVs comprises an increased production of EVs and/or a decreased degradation of EVs by the cell cultured in the CDPF medium and the lysosome inhibitor than a cell cultured without culture in the CDPF medium and the lysosome inhibitor.

In some embodiments, the culturing the cell in the CDPF medium and the lysosome inhibitor increases stability of EV production than without culturing the cell in the CDPF medium and the lysosome inhibitor. In some embodiments, the upregulated expression of CD63 comprises an increase in CD63 by the cell cultured in the CDPF medium and the lysosome inhibitor than a cell cultured without culture in the CDPF medium and the lysosome inhibitor. In some embodiments, the EVs have a mean diameter of about 85 nm to about 236 nm. In some embodiments, the cell is cultured for between about 24 hours and about 48 hours. In some embodiments, the cell is cultured to about 70˜80% confluency.

Provided herein relates to a cell capable of an enriched production of extracellular vesicles (EVs), comprising culturing the cells in a chemically-defined, protein-free (CDPF) medium and a lysosome inhibitor. In some embodiments, the cell comprises a mammalian cell having an enriched expression of CD63.

Further embodiments relate to a method for preparing mammalian cells having an enriched expression of CD63. In some embodiments, the method comprises culturing the cells in a chemically-defined, protein-free (CDPF) medium and a lysosome inhibitor. In some embodiments, the mammalian cell comprises a stem cell, an immune cell, a naïve cell, or an engineered cell. In some embodiments, the mammalian cell comprises a mesenchymal stem cell that is derived from a bone marrow, an umbilical cord, a placenta, or an adipose tissue. In some embodiments, the immune cell comprises a T cell and a NK cell. In some embodiments, CDPF medium is supplemented with additives comprising hypoxanthine, thymidine, glutamine, glucose, essential amino acids, or vitamins, or a combination thereof.

In some embodiments, the lysosome inhibitor comprises a beclin-1 inhibitor. In some embodiments, the lysosome inhibitor comprises an inhibitor of autophagosome or autophagolysosome formation. In some embodiments, the lysosome inhibitor is selected from a group consisting of SP600125, U0126, 3-methyladenine, bafilomycin A-1, LY294002, SB202190, SB203580, and chloroquine. In some embodiments, culturing the cell in the CDPF medium and the lysosome inhibitor reduces internal acidification of the cell. In some embodiments, culturing the cell in the CDPF medium and the lysosome inhibitor results in upregulation of fatty acid gene expression. In some embodiments, the fatty acid gene comprises fatty acid synthase. In some embodiments, culturing the cell in the CDPF medium and the lysosome inhibitor decreases formation of lysosomes and/or autophagosomes by the cell.

In some embodiments, the enriched production of EVs comprises at least about 2-fold, 3-fold, 4-fold, or 5-fold EVs, compared to the amount of EVs from cells cultured without CDPF medium and lysosome inhibitor. In some embodiments, the method comprises passing the conditioned culture medium over an anion exchange column to isolate EVs having a negative surface charge. In some embodiments, the EVs negative surface charge have an enriched expression of CD63. In some embodiments, the anion exchange column is a resin anion exchange column.

In additional embodiments is described a method for increasing production of extracellular vesicles (EVs). In some embodiments, the method comprises culturing mammalian cells in a chemically-defined, protein-free (CDPF) medium and a lysosome inhibitor to produce a conditioned culture medium, separating the conditioned culture medium after cell culture from the cells, and purifying EVs from the conditioned culture medium. In some embodiments, the mammalian cell comprises a stem cell, an immune cell, a naïve cell, or an engineered cell. In some embodiments, the mammalian cell comprises a mesenchymal stem cell that is derived from a bone marrow, an umbilical cord, a placenta, or an adipose tissue. In some embodiments, the immune cell comprises a T cell and a NK cell.

In some embodiments, CDPF medium is supplemented with additives comprising hypoxanthine, thymidine, glutamine, glucose, essential amino acids, or vitamins, or a combination thereof. In some embodiments, the purifying EVs comprises passing the conditioned culture medium over an anion exchange column isolate EVs having a negative surface charge. In some embodiments, passing the conditioned culture medium over an anion exchange column removes cellular debris from the conditioned culture medium. In some embodiments, the EVs negative surface charge have an enriched expression of CD63. In some embodiments, the anion exchange column is a resin anion exchange column.

The present disclosure also relates to a kit for an enriched production of extracellular vesicles (EVs). In some embodiments, the kit comprises a chemically-defined, protein-free (CDPF) medium, a lysosome inhibitor, and a mammalian cell. In some embodiments, CDPF medium is supplemented with additives comprising hypoxanthine, thymidine, glutamine, glucose, essential amino acids, or vitamins, or a combination thereof. In some embodiments, the mammalian cell comprises a stem cell, an immune cell, a naïve cell, or an engineered cell. In some embodiments, the mammalian cell comprises a mesenchymal stem cell that is derived from a bone marrow, an umbilical cord, a placenta, or an adipose tissue. In some embodiments, the immune cell comprises a T cell and a NK cell.

In some embodiments, the lysosome inhibitor comprises a beclin-1 inhibitor. In some embodiments, the lysosome inhibitor comprises an inhibitor of autophagosome or autophagolysosome formation. In some embodiments, the lysosome inhibitor is selected from a group consisting of SP600125, U0126, 3-methyladenine, bafilomycin A-1, LY294002, SB202190, SB203580, and chloroquine. The present disclosure further relates to a composition comprising extracellular vesicles (EV) produced by the cell disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic of the secretion of EVs from an adult mesenchymal stem cell (MSC) cultured in a chemically defined protein free medium (CDPF) with lysosome inhibitors.

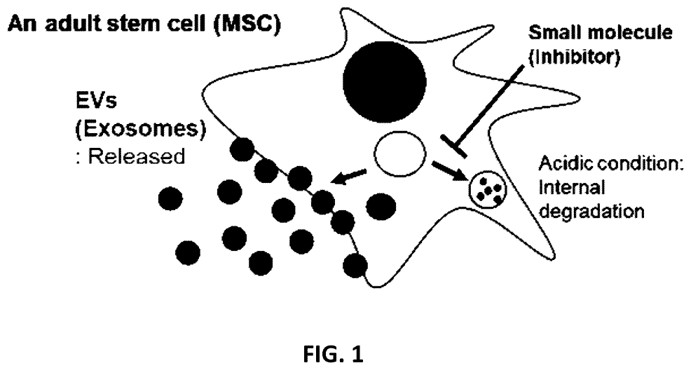

FIG. 2A shows the intracellular acidification of human mesenchymal stem/stromal cells (MSCs) in different media including complete culture medium (CCM), chemically defined protein free medium (CDPF), and CDPF with lysosome inhibitor. Lysosome inhibitor suppresses the intracellular acidification of human MSCs.

FIG. 2B shows the expression of human CD63 (hCD63) gene of the MSCs cultured in different media including CCM, CDPF, and CDPF with lysosome inhibitor as measured by qPCR. Lysosome inhibitor increased the expression of human CD63 gene in the MSCs.

FIG. 3A shows Enzyme-Linked Immunosorbent Assay (ELISA) results for CD63 of culture media collected after culture with stem cells, which indicates the secretion of EVs (CD63-positive particles) from stem cells to cultured media. CDPF with a lysosome inhibitor (Ly_Inhibitor+CDPF) stimulated the secretion of EVs (CD63-positive particles) from stem cells to the cultured media as compared to stem cells without lysosome inhibitor treatment (CCM and CDPF).

FIG. 3B shows isolation of EVs from CDPF or CDPF with lysosome inhibitor collected after culture with stem cells by anion exchange column (AIEX). Compared to that of CDPF cultured media, lysosome inhibitor-treated cultured media showed approximately 3-folds higher production yield in total as measured by CD63 ELISA of the collections fractions from AIEX.

FIG. 3C shows Quantitative Polymerase Chain Reaction (qPCR) relative quantification (RQ) for fatty acid synthase (FASN) in MSCs cultured in different media including CCM, CCM with lysosome inhibitor, CDPF, and CDPF with lysosome inhibitor. As a mode of action of the lysosome inhibitor in the EV biogenesis, treatment of the inhibitor drastically induced the gene expression of FASN, a key component of EV bilayers.

FIG. 4 shows results of anti-inflammatory activity test using 4 different lots of EVs. Macrophages (RAW 264.7) were cultured with one of the EV preparations including Lot #1 to Lot #4 at 2.5×10⁸ EVs/ml or 1.25×10⁹ EVs/ml or a positive control of dexamethasone after inflammation was induced by the addition of LPS. Among the 4 lots of EVs, EV of Lot #4 is prepared by the treatment with a lysosome inhibitor. The levels of IL-6, a proinflammatory cytokine, were determined by ELISA. All groups cultured with one of the EVs preparations showed lower IL-6 levels than the positive control, generally in a dose dependent manner.

### DETAILED DESCRIPTION

Extracellular vesicles (EVs) are typically nano-sized, lipid bilayer-delimited particles secreted from a wide variety of cell types. They are membrane-bound vesicles released from cells to the extracellular environment. Usually, subtypes of EVs include but are not limited to exosomes, microvesicles, and apoptotic bodies, which can be loaded with a wide range of therapeutic cargo such as nucleic acids (DNAs, mRNAs, miRNAs, and other non-coding RNAs), proteins, lipids, and metabolites. When EVs are taken up by recipient cells, they can deliver selectively packaged bioactive molecules (therapeutic cargo) from donor to recipient cells and initiate different intracellular effects in the cell.

In some cases, EVs may facilitate intercellular communication processes between cells in close proximity as well as distant cells. In some cases, EVs are released by immune cells. In some cases, EVs may act as antigen-presenting vesicles. In some cases, EVs may stimulate antitumoral immune responses or induce tolerogenic effects to suppress inflammation. In some cases, EVs may inactivate T lymphocytes or natural killer cells. In some cases, EVs may promote differentiation of regulatory T lymphocytes to suppress immune reactions. In some cases, EVs may participate in myelin formation, neurite outgrowth, and neuronal survival. In some cases, EVs may be capable of promoting the assembly of the enzyme complexes acting on the coagulation cascade, resulting in cell fusion events that may lead to thrombus formation. In some cases, EVs may act as both anti-inflammatory and pro-inflammatory factors depending on the stimulus that generates them and the cell from which they are released. In some cases, EVs may lead to secretion of cytokines that modulate the inflammatory response. In some cases, EVs have been thus widely explored for diagnostics, therapeutics, and biological research.

Despite the potential of EVs, there remains challenges in enriched production and isolation of high-quality EVs that have high purity, low contaminants, and/or consistent size and characteristics. Overcoming such challenges would facilitate use of EVs in various clinical applications. Often, high-quality EVs with minimized contaminants from living cells may be produced by 1) preparing EVs-enriched cell-conditioned medium, 2) isolating EVs with high quality, and/or 3) quality control testing of EVs. In some cases, the preparation of EVs enriched cell-conditioned media may be a key rate-determining step to affect both the quality and quantity of EVs. Since there may be a lack of consensus regarding the preparation and isolation steps of high-quality EVs, various methods to retrieve EVs from biofluids or cell-cultured media have been reported. To prepare EVs with high quality from a cell-conditioned medium, there are several conditions should be considered, including but not limited to efficiency of EV production and isolation, reproducibility of the EV production process, and low toxicity of the produced EVs.

Disclosed herein are solutions to these and other problems known in the art. The present disclosure provides methods, systems, cells, and kits for an enriched production of high-quality EVs from various cell sources, including but not limited to mammalian cells. In some cases, the methods may comprise culturing the cell in a chemically-defined protein-free (CDPF) medium with the addition of a lysosome inhibitor to produce a conditioned culture medium comprising EVs produced and released by the cells. In some cases, the CDPF medium is supplemented with additives to facilitate the production of EVs by the cells. In some cases, the additives comprise a lysosome inhibitor. In some cases, the addition of a lysosome inhibitor to the CDPF may enhance EV production by the cultured cells. . In some cases, the addition of a lysosome inhibitor to the CDPF may facilitate production of higher quality EVs. In some case, higher quality EVs comprise EVs that more consistent in size, composition, and other characteristics across different batches of cell sources. In some cases, culturing the cells in the CDPF medium with the addition of a lysosome inhibitor may result in an upregulated expression of CD63 by the cells. In some cases, culturing the cell in the CDPF medium with the addition of the lysosome inhibitor may result in the upregulation of fatty acid gene expression. In some cases, the lysosome inhibitor may reduce the internal acidification of the cell. In some cases, the enriched production of high-quality EVs comprises at least about 3-fold EVs, compared to the amount of EVs from cells cultured without CDPF medium and lysosome inhibitor.

### I. DEFINITIONS

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” means a range of values including the specified value, which a person of ordinary skill in the art would consider reasonably similar to the specified value. In some embodiments, about means within a standard deviation using measurements generally acceptable in the art. In some embodiments, about means a range extending to +/−10% of the specified value. In some embodiments, about includes the specified value.

In this disclosure, “comprises,” “comprising,” “containing” and “having” and the like can have the meaning ascribed to them in U.S. Patent law and can mean “ includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### II. CELL

Usually, EVs are released by many types of cell. Cells produce different types of EVs, for example, that vary in size. In some cases, EVs may deliver nucleic acid, proteins, or lipids that can be functional in recipient cells. In some cases, EV membranes may consist of a lipid bilayer similar to that of cell plasma membrane. In some cases, the amount of delivered product in EVs may be controlled by the steps used in the EV production process.

As used herein, a “cell” generally refers to a mammalian cell. A cell can be the basic structural, functional and/or biological unit of a living organism. A cell can originate from any organism having one or more cells. Some non-limiting examples include: a stem cell, an immune cell, a naïve (natural) cell, a prokaryotic cell, and an engineered cell. In some embodiments, the stem cell comprises a mesenchymal stem cell that can be found in adult tissue (e.g., muscle, liver, or bone marrow). In some embodiments, the stem cell comprises an adult stem cell, an embryonic stem cell, a mesenchymal stem cell, or an induced pluripotent stem cell (iPS), or a combination thereof. In some embodiments, the mesenchymal stem cell comprises cells derived from bone marrow, a placenta, an umbilical cord, adipose tissue, skeletal muscle tissue, skin tissue, tooth tissues, gum tissue, brain tissue, heart tissue, intestinal tissue, liver tissue, spinal cord tissue, cord blood, peripheral blood, blood vessels, ovarian epithelium, umbilical cord tissue, amniotic fluid, or testicular tissue. In some embodiments, the stem cell comprises a totipotent, pluripotent, multipotent, oligopotent, or unipotent cell, or a combination thereof. In some embodiments, the immune cell comprises lymphocytes, neutrophils, or monocytes/macrophages, or a combination thereof. In some embodiments, the immune cell comprises a T cell, a B cell, and an NK cell. In some embodiments, the naïve (natural) cell comprises natural immune cells which are non-engineered immune cells, e.g., cells prepared from blood or tissues. In some embodiments, the engineered cell comprises artificially generated immune cells from natural immune cells (e.g., Chimeric antigen receptor T cells, Uni-CAR T-cells, and subtypes thereof) using gene or protein engineering technology. In some embodiments, the engineered cell comprises an engineered derivatives from the naïve (natural) cell comprising a stem cell, an immune cell, a probiotics, or 293 embryonic kidney cell. In some embodiments, the engineered cell comprises engineered T-cell line or engineered NK-cell line. In some embodiments, the engineered T-cell line comprises CAR-T cells. In some embodiments, the engineered NK cell line comprises Uni-CAR cells. In some embodiments, the prokaryotic cell comprises probiotics comprising a Lactobacillus, a Bifidobacterium, and the like. In some embodiments, a cell may be selected from established cell lines, including but not limited to 293 embryonic kidney cells.

In some embodiments, a naïve (natural) cell-derived EV comprises natural EVs isolated from non-engineered cells like mesenchymal stem cells, neural stem cells, embryonic stem cells, immune cells, and the like. In some embodiments, an engineered cell-derived EV comprises artificially engineered EVs isolated from cargo (mRNA, proteins, peptides, and the like) loading inside/outside cells by using engineering technology like exosomal protein anchoring, protein-protein interaction, peptide targeting, and the like.

In some embodiments, the cells used in the methods, systems, and kits provided herein can produce extracellular vesicles. In some embodiments, the cells can express CD63. In some embodiments, the cells can produce extracellular vesicles comprising CD63 markers.

### III. CELL CULTURE MEDIA

For the preparation of EVs by cell culture, culture media supplemented with serum and/or cytokines and/or hypoxia-conditioned culture condition are commonly used. Major issues in these protocols include but are not limited to internal acidification of cells, instability of EV production, and donor to donor variation, which may adversely affect the quality and quantity of EVs that are produced by the cells. To obtain high-quality EVs, it is necessary to consider an appropriate culture medium, an optimized cell density, a cell phenotype, culture time, collection time, and other parameters.

The methods, systems, cells, and kits described herein are directed to culturing a cell in a chemically-defined protein-free (CDPF) medium with the addition of a lysosome inhibitor to increase the EVs production and to decrease the amounts of contaminants (e.g., serum, impurities, unassociated-EV molecules, and the like). Compared to complete cell culture medium (CCM), the CDPF medium is serum free and does not include proteins. In some cases, the CDPF medium with a lysosome inhibitor may be used for culturing various mammalian cells to generate EVs. In some cases, the CDPF medium with a lysosome inhibitor is used for culturing human mesenchymal stem cells. In some cases, the CDPF medium with a lysosome inhibitor is supplemented with a range of non-protein additives. In some cases, the CDPF medium and lysosome inhibitor comprises a combination of ingredients disclosed herein. In some cases, the non-protein additives comprise hypoxanthine, thymidine, glutamine, glucose, essential amino acids, or vitamins, or a combination thereof. In some cases, lysosome inhibitors are added to CDPF media in the process of production of EVs to reduce internal acidification of the cell. In some cases, culturing the cell in the CDPF medium and the lysosome inhibitor decreases formation of lysosomes and/or autophagosomes by the cell. In some cases, culturing the cell in the CDPF medium and the lysosome inhibitor results in upregulation of fatty acid gene expression. In some embodiments, the fatty acid gene comprises fatty acid synthase. In some embodiments, the lysosome inhibitor comprises a beclin-1 inhibitor. In some embodiments, the lysosome inhibitor comprises an inhibitor of autophagosome or autophagolysosome formation.

Table 1 shows an exemplary composition of the CDPF medium. In some embodiments, the CDPF medium comprises CD CHO protein free media having a working concentration of about 850 ml/L, about 875 ml/L, about 900 ml/L, about 925 ml/L, about 950 ml/L, about 975 ml/L, or about 1000 ml/L. In some embodiments, the CDPF medium comprises CD CHO protein free media having a working concentration of between about 850 ml/L and about 1000 ml/L, between about 875 ml/L and about 975 ml/L, between about 900 ml/L and about 950 ml/L, or between about 925 ml/L and about 975 ml/L. In some embodiments, the CDPF medium comprises HT supplement having a working concentration of about 5 ml/L, about 6 ml/L, about 7 ml/L, about 8 ml/L, about 9 ml/L, about 10 ml/L, about 11 ml/L, about 12 ml/L, about 13 ml/L, about 14 ml/L, or about 15 ml/L. In some embodiments, the CDPF medium comprises HT supplement having a working concentration of between about 5 ml/L and about 15 ml/L, between about 6 ml/L and about 14 ml/L, between about 7 ml/L and about 13 ml/L, between about 8 ml/L and about 12 ml/L, between about 9 ml/L and about 11 ml/L, or between about 10 ml/L and about 15 ml/L. In some embodiments, the CDPF medium comprises 200 mL L-glutamine having a working concentration of about 30 ml/L, about 35 ml/L, about 40 ml/L, about 45 ml/L, or about 50 ml/L. In some embodiments, the CDPF medium comprises 200 mL L-glutamine having a working concentration of between about 30 ml/L and about 50 ml/L, between about 35 ml/L and about 45 ml/L, or between about 40 ml/L and about 50 ml/L. In some embodiments, the CDPF medium comprises D-(+)-glucose having a working concentration of about 0.5 g, about 1 g, about 1.5 g, about 2 g, about 2.5 g, or about 3 g. In some embodiments, the CDPF medium comprises D-(+)-glucose having a working concentration of between about 0.5 g and about 3 g, between about 1 g and about 2.5 g, between about 1.5 g and about 2 g, or between about 2 g and about 3 g. In some embodiments, the CDPF medium comprises 100× nonessential amino acid having a working concentration of about 5 ml/L, about 10 ml/L, about 15 ml/L, about 20 ml/L, or about 25 ml/L. In some embodiments, the CDPF medium comprises 100× nonessential amino acid having a working concentration of between about 5 ml/L and about 25 ml/L, between about 10 ml/L and about 20 ml/L, or between about 15 ml/L and about 25 ml/L. In some embodiments, the CDPF medium comprises 100× MEM vitamin solution having a working concentration of about 5 ml/L, about 10 ml/L, about 15 ml/L, about 20 ml/L, or about 25 ml/L. In some embodiments, the CDPF medium comprises 100× MEM vitamin solution having a working concentration of between about 5 ml/L and about 25 ml/L, between about 10 ml/L and about 20 ml/L, or between about 15 ml/L and about 25 ml/L.

TABLE 1
Exemplary composition of CDPF medium

| Additives | Working concentration (ml/L) |
|---|---|
| CD CHO protein free media | 850-1000 |
| HT supplement | 5-15 |
| 200 mL L-Glutamine | 30-50 |
| D-(+)-Glucose | 0.5-3 g |
| 100x nonessential amino acid | 5-25 |
| 100x MEM vitamin solution | 5-25 |

### IV. LYSOSOME INHIBITOR

In some embodiments, the use of serum-reduced or -free media in culturing cells to generate EVs may result in undesired effects that adversely affect EV production. In some embodiments, these culture conditions may generally arrest most of the cell's growth to a specific stage of the cell cycle and induce intracellular acidification of cells. In some embodiments, the intracellular acidification may result in the degradation of cell components. In some embodiments, the addition of a lysosome inhibitor may reduce or inhibit intracellular acidification of being cultured cells, resulting in decreasing the formation of lysosomes and autophagosomes which are involved in the lysis process of cell components. The methods disclosed herein are directed to the addition of lysosome inhibitors to CDPF media used to culture cells in the process of production of EVs to reduce internal acidification in the cells. In some cases, culturing the cell in the CDPF medium and the lysosome inhibitor decreases or inhibits formation of lysosomes and/or autophagosomes by the cell. In some embodiments, the lysosome inhibitor comprises a beclin-1 inhibitor. In some embodiments, the lysosome inhibitor comprises an inhibitor of autophagosome or autophagolysosome formation.

In some embodiments described herein, lysosome inhibitors are added to CDPF media in the process of production of EVs to prepare high-quality EVs with high yields. In some embodiments, the preparation of high-quality EVs with high efficiencies comprises an increased production of EVs and/or a decreased degradation of EVs by the cell cultured in the CDPF medium and the lysosome inhibitor than a cell cultured without culture in the CDPF medium and the lysosome inhibitor. In some cases, culturing the cell in the CDPF medium and the lysosome inhibitor increases activities of EVs than without culturing the cell in the CDPF medium and the lysosome inhibitor. In some cases, culturing the cell in the CDPF medium and the lysosome inhibitor upregulates expression of CD63 than without culturing the cell in the CDPF medium and the lysosome inhibitor. In some cases, culturing the cell in the CDPF medium and the lysosome inhibitor results in upregulation of fatty acid gene expression than without culturing the cell in the CDPF medium and the lysosome inhibitor.

In some embodiments, the lysosome inhibitor comprises an inhibitor of beclin-1. In some embodiments, the lysosome inhibitor comprises an inhibitor of autophagosome/autophagolysosome formation. In some embodiments, the beclin-1 inhibitor comprises SP600125 or U0126 or a combination thereof. In some embodiments, an inhibitor of autophagosome/autophagolysosome formation comprises 3-methyladenine, bafilomycin A-1, LY294002, SB202190, SB203580, wortmannin, or chloroquine or a combination thereof. In some cases, the lysosome inhibitor reduces the internal acidification of cells. In some cases, culturing cells in the CDPF medium and the lysosome inhibitor results in the upregulation of fatty acid gene expression. In some cases, the fatty acid gene comprises fatty acid synthase. In some cases, fatty acids are one of EV markers and have anti-inflammatory potential in acute/chronic diseases comprising, but not limiting to, cardiovascular diseases, inflammatory bowel disease (IBD), cancer, and rheumatoid arthritis. In some cases, the anti-inflammatory potential comprises a reduction in inflammation in the subject. In some cases, culturing cells in the CDPF medium and the lysosome inhibitor decreases the formation of lysosomes and/or autophagosomes by the cells.

In some embodiments, CDPF medium comprises 3-methyladenine of about 20 mM, about 30 mM, about 40 mM, about 50 mM, about 60 mM, about 70 mM, or about 80 mM. In some embodiments, CDPF medium comprises 3-methyladenine of at least about 20 mM, about 30 mM, about 40 mM, about 50 mM, about 60 mM, about 70 mM, or about 80 mM. In some embodiments, CDPF medium comprises 3-methyladenine of at most about 20 mM, about 30 mM, about 40 mM, about 50 mM, about 60 mM, about 70 mM, or about 80 mM. In some embodiments, CDPF medium comprises 3-methyladenine of between about 20 mM and about 80 mM, between about 30 mM and about 70 mM, between about 40 mM and about 60 mM, or about 50 mM and about 80 mM. In some embodiments, CDPF medium comprises bafilomycin A-1 of about 0.5 μM, about 1 μM, about 1.5 μM, about 2 μM, about 2.5 μM, or about 3 μM. In some embodiments, CDPF medium comprises bafilomycin A-1 of at least about 0.5 μM, about 1 μM, about 1.5 μM, about 2 μM, about 2.5 μM, or about 3 μM. In some embodiments, CDPF medium comprises bafilomycin A-1 of at most about 0.5 μM, about 1 μM, about 1.5 μM, about 2 μM, about 2.5 μM, or about 3 μM. In some embodiments, CDPF medium comprises bafilomycin A-1 of between about 0.5 μM and about 3 μM, between about 1 μM and about 2.5 μM, between about 1.5 μM and about 2 μM, or about 1.5 μM and about 3 μM. In some embodiments, CDPF medium comprises LY294002 of about 50 μM, about 100 μM, about 150 μM, or about 200 μM. In some embodiments, CDPF medium comprises LY294002 of at least about 50 μM, about 100 μM, about 150 μM, or about 200 μM. In some embodiments, CDPF medium comprises LY294002 of at most about 50 μM, about 100 μM, about 150 μM, or about 200 μM. In some embodiments, CDPF medium comprises LY294002 of between about 50 μM and about 200 μM, between about 100 μM and about 150 μM, or between about 50 μM and about 100 μM. In some embodiments, CDPF medium comprises SB202190 of about 50 μM, about 100 μM, about 150 μM, or about 200 μM. In some embodiments, CDPF medium comprises SB202190 of at least about 50 μM, about 100 μM, about 150 μM, or about 200 μM. In some embodiments, CDPF medium comprises SB202190 of at most about 50 μM, about 100 μM, about 150 μM, or about 200 μM. In some embodiments, CDPF medium comprises SB202190 of between about 50 μM and about 200 μM, between about 100 μM and about 150 μM, or between about 50 μM and about 100 μM. In some embodiments, CDPF medium comprises SB203580 of about 2 μM, about 4 μM, about 6 μM, about 8 μM, about 10 μM, about 12 μM, about 14 μM, about 16 μM, about 18 μM, or about 20 μM. In some embodiments, CDPF medium comprises SB203580 of at least about 2 μM, about 4 μM, about 6 μM, about 8 μM, about 10 μM, about 12 μM, about 14 μM, about 16 μM, about 18 μM, or about 20 μM. In some embodiments, CDPF medium comprises SB203580 of at most about 2 μM, about 4 μM, about 6 μM, about 8 μM, about 10 μM, about 12 μM, about 14 μM, about 16 μM, about 18 μM, or about 20 μM. In some embodiments, CDPF medium comprises SB203580 of between about 2 μM and about 20 μM, between about 4 μM and about 18 μM, between about 6 μM and about 16 μM, between about 8 μM and about 14 μM, between about 10 μM and about 12 μM, or between about 8 μM and about 20 μM. In some embodiments, CDPF medium comprises wortmannin of about 0.2 μM, about 0.4 μM, about 0.6 μM, about 0.8 μM, about 1.0 μM, about 1.2 μM, about 1.4 μM, about 1.6 μM, about 1.8 μM, or about 2.0 μM. In some embodiments, CDPF medium comprises wortmannin of at least about 0.2 μM, about 0.4 μM, about 0.6 μM, about 0.8 μM, about 1.0 μM, about 1.2 μM, about 1.4 μM, about 1.6 μM, about 1.8 μM, or about 2.0 μM. In some embodiments, CDPF medium comprises wortmannin of at most about 0.2 μM, about 0.4 μM, about 0.6 μM, about 0.8 μM, about 1.0 μM, about 1.2 μM, about 1.4 μM, about 1.6 μM, about 1.8 μM, or about 2.0 μM. In some embodiments, CDPF medium comprises wortmannin of between about 0.2 μM and about 2.0 μM, between about 0.4 μM and about 1.8 μM, between about 0.6 μM and about 1.6 μM, between about 0.8 μM and about 1.4 μM, between about 1.0 μM and about 1.2 μM, or between about 0.8 μM and about 2.0 μM. In some embodiments, CDPF medium comprises chloroquine of about 50 μM, about 100 μM, about 150 μM, or about 200 μM. In some embodiments, CDPF medium comprises chloroquine of at least about 50 μM, about 100 μM, about 150 μM, or about 200 μM. In some embodiments, CDPF medium comprises chloroquine of at most about 50 μM, about 100 μM, about 150 μM, or about 200 μM. In some embodiments, CDPF medium comprises chloroquine of between about 50 μM and about 200 μM, between about 100 μM and about 150 μM, or between about 50 μM and about 100 μM. In some embodiments, CDPF medium comprises SP600125 of about 150 μM, about 200 μM, about 250 μM, or about 300 μM. In some embodiments, CDPF medium comprises SP600125 of at least about 150 μM, about 200 μM, about 250 μM, or about 300 μM. In some embodiments, CDPF medium comprises SP600125 of at most about 150 μM, about 200 μM, about 250 μM, or about 300 μM. In some embodiments, CDPF medium comprises SP600125 of between about 150 μM and about 300 μM, between about 200 μM and about 250 μM, or between about 150 μM and about 300 μM. In some embodiments, CDPF medium comprises U0126 of about 10 μM, about 15 μM, about 20 μM, about 25 μM, or about 30 μM. In some embodiments, CDPF medium comprises U0126 of at least about 10 μM, about 15 μM, about 20 μM, about 25 μM, or about 30 μM. In some embodiments, CDPF medium comprises U0126 of at most about 10 μM, about 15 μM, about 20 μM, about 25 μM, or about 30 μM. In some embodiments, CDPF medium comprises U0126 of between about 10 μM and about 30 μM, between about 15 μM and about 25 μM, between about 20 μM and about 25 μM, or between about 15 μM and about 30 μM.

TABLE 2
Exemplary compositions of lysosome inhibitors in CDPF medium

| Additives | Working concentrations (μM) |
|---|---|
| 3-methyladenine | 20 mM-80 mM |
| bafilomycin A-1 | 0.5-3 |
| LY294002 | 50-200 |
| SB202190 | 50-200 |
| SB203580 | 2-20 |
| wortmannin | 0.2-2 |
| chloroquine | 50-200 |
| SP600125 | 150-300 |
| U0126 | 10-30 |

### V. ENRICHED PRODUCTION OF EXTRACELLULAR VESICLES

The present disclosure provides methods, systems, cells, and kits for an enriched production of high-quality EVs from a mammalian cell. In some cases, EVs include but are not limited to exosomes, microvesicles, and apoptotic bodies, which can be loaded with a wide range of therapeutic cargo such as nucleic acids (DNAs, mRNAs, miRNAs, and other non-coding RNAs), proteins, lipids, and metabolites. Exosomes are nanometer-sized vesicles (40-120 nm) of endocytic origin that form by inward budding of the limiting membrane of multivesicular endosomes. In some cases, exosomes may be enriched in endosome-associated proteins. In some cases, the endosome-associated proteins comprise but are not limited to Rab GTPases, SNAREs, Annexins, or flotillin. In some cases, tetraspanins (e.g. CD63, CD81, CD9) may be abundant in exosomes and may be considered to be exosome markers. Often, microvesicles bud from cell surface. In some cases, the size of microvesicles may vary between 50 nm to 1000 nm. In some cases, selectins, integrins, or CD40 ligand may be considered as a microvesicle marker. Dying cells release vesicular apoptotic bodies under specific conditions.

The methods comprise culturing the cell in a CDPF medium with the addition of a lysosome inhibitor to produce a conditioned culture medium. In some cases, the CDPF medium is supplemented with additives. In some cases, culturing the cell in the CDPF medium with the addition of a lysosome inhibitor may result in an upregulated expression of CD63. In some cases, culturing the cell in the CDPF medium with the addition of the lysosome inhibitor may result in the upregulation of fatty acid gene expression. In some cases, the lysosome inhibitor may reduce the internal acidification of the cell. In some cases, the enriched production of high-quality EVs comprises at least about 3-fold EVs, compared to the amount of EVs from cells cultured without CDPF medium and lysosome inhibitor.

In some embodiments, an enriched production of EVs, as used herein, may be defined as production that comprises a greater number of EV particles prepared under defined conditions by culturing mammalian cells in the CDPF medium and lysosome inhibitor, compared to the number of EV particles prepared by culturing mammalian cells without both the CDPF medium and lysosome inhibitor. In some embodiments, the enriched production of EVs comprises at least about 1.5-fold, 2-fold, 3-fold, 4-fold, or 5-fold higher in EV quantity (the number of EV particles), compared to the amount of EVs from cells cultured without CDPF medium and lysosome inhibitor. In some embodiments, the enriched production of EVs comprises at least about 2-fold higher in EV quantity, compared to the amount of EVs from cells cultured without CDPF medium and lysosome inhibitor. In some embodiments, the enriched production of EVs comprises at least about 3-fold higher in EV quantity, compared to the amount of EVs from cells cultured without CDPF medium and lysosome inhibitor. In some embodiments, the enriched production of EVs comprises at least about 4-fold higher in EV quantity, compared to the amount of EVs from cells cultured without CDPF medium and lysosome inhibitor. In some embodiments, the enriched production of EVs comprises at least about 5-fold higher in EV quantity, compared to the amount of EVs from cells cultured without CDPF medium and lysosome inhibitor.

In some embodiments, an enriched production of EVs, as used herein, may be defined as production that comprises a higher anti-inflammatory activities of EVs prepared under defined conditions by culturing mammalian cells in the CDPF medium and lysosome inhibitor, compared to the anti-inflammatory activities of EVs prepared by culturing mammalian cells without both the CDPF medium and lysosome inhibitor. In some embodiments, the enriched production of EVs comprises at least about 3-fold, 4-fold, 5-fold, 6-fold, or 7-fold higher anti-inflammatory activities of EVs, compared to the EVs from cells cultured without CDPF medium and lysosome inhibitor. In some embodiments, the enriched production of EVs comprises at least about 3-fold higher anti-inflammatory activities of EVs, compared to the amount of EVs from cells cultured without CDPF medium and lysosome inhibitor. In some embodiments, the enriched production of EVs comprises at least about 4-fold higher anti-inflammatory activities of EVs, compared to the amount of EVs from cells cultured without CDPF medium and lysosome inhibitor. In some embodiments, the enriched production of EVs comprises at least about 5-fold higher anti-inflammatory activities of EVs, compared to the amount of EVs from cells cultured without CDPF medium and lysosome inhibitor. In some embodiments, the enriched production of EVs comprises at least about 6-fold higher anti-inflammatory activities of EVs, compared to the amount of EVs from cells cultured without CDPF medium and lysosome inhibitor. In some embodiments, the enriched production of EVs comprises at least about 7-fold higher anti-inflammatory activities of EVs, compared to the amount of EVs from cells cultured without CDPF medium and lysosome inhibitor.

In some embodiments, the enriched production of EVs comprises EVs with a mean diameter of about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, or about 280 nm. In some embodiments, the enriched production of EVs comprises EVs with a mean diameter of between about 70 nm and about 280 nm, between about 80 nm and about 270 nm, between about 90 nm and about 260 nm, between about 100 nm and about 250 nm, between about 110 nm and about 240 nm, between about 120 nm and about 230 nm, between about 130 nm and about 220 nm, between about 140 nm and about 210 nm, between about 150 nm and about 200 nm, between about 160 nm and about 190 nm, or between about 170 nm and about 180 nm. In some embodiments, the enriched production of EVs comprises EVs with a mean diameter of about 85 nm to about 236 nm.

In some embodiments, various measurements may serve as markers of enriched production of EVs by the cultured cells as described herein. In some embodiments, the enriched production of EVs may be indicated as an upregulated expression of CD63. In some embodiments, the enriched production of EVs may be indicated as upregulation of fatty acid gene expression. In some embodiments, the fatty acid gene comprises fatty acid synthase. In some embodiments, the enriched production of EVs may be indicated as reducing internal acidification of the cell. In some embodiments, the enriched production of EVs may be indicated as decreasing the formation of lysosomes and/or autophagosomes by the cell. In some embodiments, the enhanced production of EVs comprises an increased production of EVs and/or a decreased degradation of EVs by the cell cultured in the CDPF medium and the lysosome inhibitor than a cell-cultured without culture in the CDPF medium and the lysosome inhibitor. In some embodiments, the culturing of the cell in the CDPF medium and the lysosome inhibitor increases the stability of EV production, compared to without culturing the cell in the CDPF medium and the lysosome inhibitor. In some embodiments, the upregulated expression of CD63 comprises an increase in CD63 by the cell cultured in the CDPF medium and the lysosome inhibitor than a cell-cultured without culture in the CDPF medium and the lysosome inhibitor.

For an enriched production of EVs, it is important to consider an appropriate culture medium, an optimized cell density, a cell phenotype, culture time, collection time, and other parameters. In some cases, the enriched production of EVs comprises culturing cells in the CDPF medium and lysosome inhibitor for between about 24 hours and about 84 hours. In some cases, the enriched production of EVs comprises culturing cells in the CDPF medium and lysosome inhibitor for at least about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, or about 84 hours. In some cases, the enriched production of EVs comprises culturing cells in the CDPF medium and lysosome inhibitor between about 18 hours and about 84 hours, between about 20 hours and about 82 hours, between about 22 hours and about 80 hours, between about 24 hours and about 78 hours, between about 26 hours and about 76 hours, between about 28 hours and about 74 hours, between about 30 hours and about 72 hours, between about 32 hours and about 70 hours, between about 34 hours and about 68 hours, between about 36 hours and about 66 hours, between about 38 hours and about 64 hours, between about 40 hours and about 62 hours, between about 42 hours and about 60 hours, between about 44 hours and about 58 hours, between about 46 hours and about 56 hours, between about 48 hours and about 54 hours, between about 50 hours and about 52 hours, or between about 28 hours and about 82 hours.

In some embodiments, the enriched production of EVs comprises seeding the cells at a density of 5,000 cells/cm² and culturing cells in a CDPF medium and a lysosome inhibitor until reaching about 70-80% cell density that occupy the entire culture area before the conditioned medium is collected. In some embodiments, the cells are seeded at a density of about 2,500 cells/cm², 5,000 cells/cm², 7,500 cells/cm², or 10,000 cells/cm². In some embodiments, the cell is cultured to about 65-90% confluency before the conditioned medium is collected. In some embodiments, the cell is cultured to about 70-80% confluency before the conditioned medium is collected. In some embodiments, the cell is cultured to about 65%, about 70%, about 75%, about 80%, about 85%, or about 90% confluency before the conditioned medium is collected.

### VI. OTHER CONDITIONS

In further embodiments, the method of an enriched production of EVs from mammalian cells cultured in the CDPF medium and lysosome inhibitor comprises culturing mammalian cells in a CDPF medium and a lysosome inhibitor to produce a conditioned culture medium, separating the conditioned culture medium after cell culture from the cells, and purifying EVs from the conditioned culture medium. In some embodiments, the method of an enriched production of EVs from mammalian cells cultured in the CDPF medium and lysosome inhibitor comprises passing the conditioned culture medium over an anion exchange column to isolate EVs having a negative surface charge. In some embodiments, passing the conditioned culture medium over an anion exchange column removes cellular debris from the conditioned culture medium. In some embodiments, the EVs negative surface charge have an enriched expression of CD63. In some embodiments, the anion exchange column is a resin anion exchange column.

An anion exchange column contains positively charged packing material and therefore retains negatively charged molecules by coulombic interaction. In some embodiments, packing material of an anion exchange column is a quaternary amine chromatography material or a tertiary amine chromatography material. In some embodiments, pH gradient inside the column is a linear gradient. In some embodiments, the pH gradient inside the column is a step gradient. In some embodiments, the pH gradient inside the column comprises a decrease from about pH 8 to about pH 5. In some embodiments, the pH gradient inside the column is generated using one or more buffers. In other embodiments, the one or more buffers is piperazine, imidazole or Tris.

### VII. KITS

Additional aspect of the present disclosure provides a kit for an enriched production of EVs. In some cases, the kit comprises a CDPF medium, a lysosome inhibitor, and a mammalian cell. The kits described herein are directed to culturing a cell in a CDPF medium with the addition of a lysosome inhibitor to increase the EVs production and to decrease the amounts of contaminants (e.g., serum, impurities, unassociated-EV molecules, and the like). Compared to complete cell culture medium (CCM), the CDPF medium is serum free and does not include proteins. In some cases, the CDPF medium is supplemented with additives comprising hypoxanthine, thymidine, glutamine, glucose, essential amino acids, or vitamins, or a combination thereof. In some cases, the lysosome inhibitor may induce an upregulated expression of CD63. In some cases, the lysosome inhibitor may induce the upregulation of fatty acid gene expression. In some cases, the lysosome inhibitor may reduce the internal acidification of the cell, resulting in decreasing the formation of lysosomes and autophagosomes which are involved in the lysis process of cell components. In some cases, the lysosome inhibitor comprises a beclin-1 inhibitor. In some cases, the lysosome inhibitor comprises an inhibitor of autophagosome or autophagolysosome formation. In some cases, the lysosome inhibitor is selected from a group consisting of SP600125, U0126, 3-methyladenine, bafilomycin A-1, LY294002, SB202190, SB203580, and chloroquine.

In some cases, the mammalian cell comprises a stem cell, an immune cell, a naïve cell, or an engineered cell. In some cases, the mammalian cell comprises a mesenchymal stem cell that is derived from a bone marrow, an umbilical cord, a placenta, or an adipose tissue. In some embodiments, the immune cell comprises a T cell, a B cell, and an NK cell. In some embodiments, the naïve (natural) cell comprises natural immune cells which are non-engineered immune cells, e.g., cells prepared from blood or tissues. In some embodiments, the engineered cell comprises artificially generated immune cells from natural immune cells (e.g., Chimeric antigen receptor T cells, UniCAR T-cells, and subtypes thereof) using gene or protein engineering technology.

### VIII. EXAMPLES

The following examples are included for illustrative purposes only and are not intended to limit the scope of the invention.

### Example 1: Chemically Defined Protein Free (CDPF) Medium and Lysosome Inhibitor for Culturing Mesenchymal Stem Cells (MSCs)

The example provides an exemplary composition of the chemically defined protein free medium with the addition of lysosome inhibitor and non-protein additives for culturing human mesenchymal stem cells. The composition disclosed herein is to activate human MSCs resulting in enriched production of EVs.

The composition of CDPF medium with the addition of lysosome inhibitor (chloroquine) and non-protein additives are shown in Table 3:

TABLE 3
CDPF media with lysosome inhibitor for
preparation of human MSC-derived EVs

| Components | Working concentration (ml/L) |
|---|---|
| CD CHO protein free media | 850-1000 |
| HT supplement | 5-15 |
| 200 mL L-Glutamine | 30-50 |
| D-(+)-Glucose | 0.5-3 g |
| 100x nonessential amino acid | 5-25 |
| 100x MEM vitamin solution | 5-25 |

### Example 2: Lysosome Inhibitor Suppresses the Intracellular Acidification of Human MSCs and Increases the Expression of Human CD63+ Genes

Mesenchymal stem cells were seeded on the plate at a density of ˜5000 cells/cm² and the plate was placed in 5% CO₂ humidified incubator. One day after the incubation, the culture media was aspirated to remove non-adherent cells and switched with a fresh media. The plate was incubated again. After further culturing for around 3 to 5 days, the culture media was aspirated and replaced by CDPF and/or lysosome inhibitor medium. The cells were cultured in the CDPF and/or lysosome inhibitor medium for 12, 24, or 48 hours. After the designated period of culturing, the culture media was collected and cell lysis buffer was immediately added to the cells on the plate. The collected media and cell lysate were each independently subjected to detection of the level of CD63 by ELISA and by qPCR, respectively.

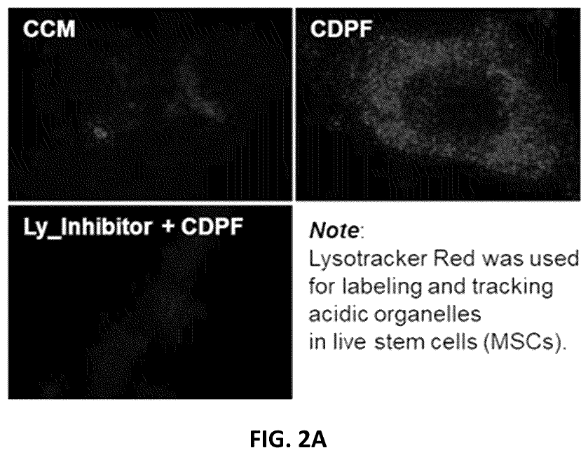

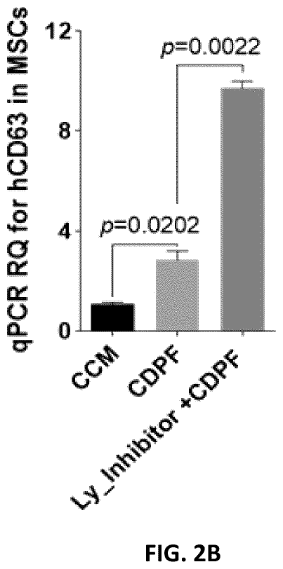

Human MSCs were cultured in different media including complete culture medium (CCM), CDPF, and CDPF with lysosome inhibitor. The acidic organelles in live stem cells were stained by lysotracker red. As shown in FIG. 2A, the intracellular acidification of human MSCs was suppressed by culturing MSCs in CDPF and lysosome inhibitor. Without lysosome inhibitor, the intracellular acidification was not suppressed.

To compare hCD63+ expression in MSCs cultured in various media including CCM, CDPF, and CDPF with lysosome inhibitor, RT-quantitative real-time PCR (RT-qPCR) assays were performed. FIG. 2B demonstrates a dramatic increase in CD63+ gene expression by culturing MSCs in CDPF and lysosome inhibitor. Using RT-qPCR, the expression of hCD63+ gene were about 10 times higher for MSCs cultured in CDPF with lysosome inhibitor than that of MSCs cultured in CCM and about 5 times higher than of that of MSCs cultured in CDPF without the addition of lysosome inhibitor.

### Example 3: Lysosome Inhibitor Stimulates the Secretion of EVs From Stem Cells to Cultured Media

Mesenchymal stem cells were seeded on the plate at a density of 5000 cells/cm² and the plate was placed in 5% CO₂ humidified incubator. One day after the incubation, the culture media was aspirated to remove non-adherent cells and switched with a fresh media. The plate was incubated again. After further culturing for around 3 to 5 days, the culture media was aspirated and replaced by CDPF and/or lysosome inhibitor medium. The cells were cultured in the CDPF and/or lysosome inhibitor medium for 12, 24, or 48 hours. After the designated period of culturing, the culture media was collected and cell lysis buffer was immediately added to the cells on the plate. The collected media and cell lysate were each independently subjected to detection of the level of CD63 by ELISA and by qPCR, respectively.

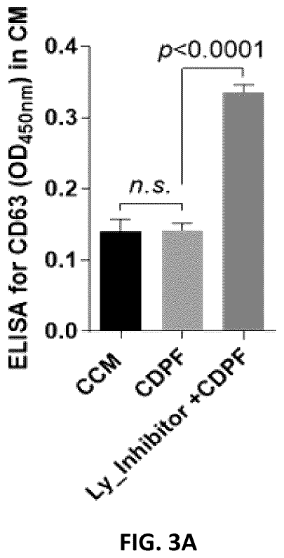

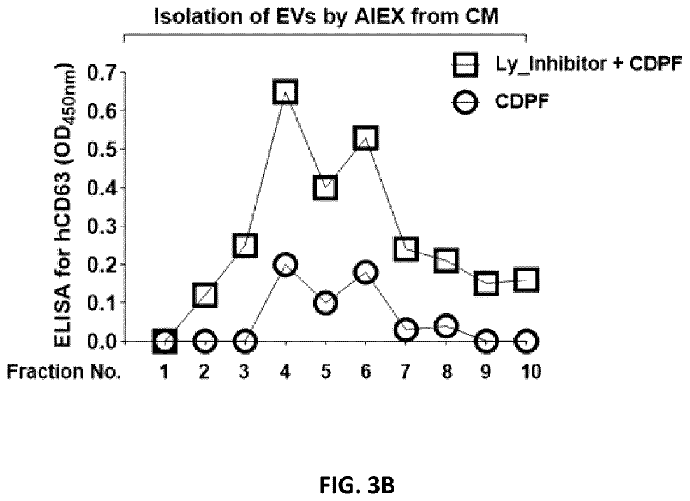

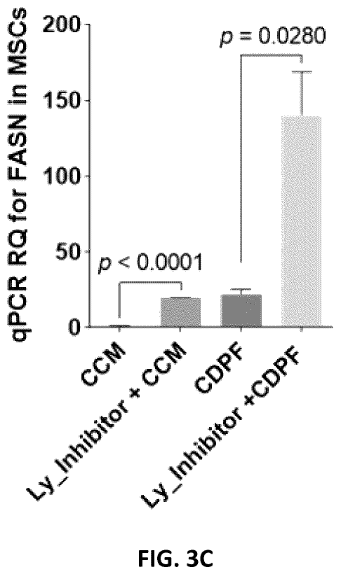

To quantitatively compare CD63+ expression in CM cultured in various media including CCM, CDPF, and CDPF with lysosome inhibitor, enzyme-linked immunosorbent (ELISA) assays were performed. As shown in FIG. 3A, culturing human MSCs in CDPF and lysosome inhibitor stimulated the secretion of EVs (CD63+ particles) from stem cells to cultured medium. Using ELISA, the EV productions were about 2.3 times higher for MSCs cultured in CDPF with lysosome inhibitor than that of MSCs cultured in CCM or in CDPF without the addition of lysosome inhibitor. Compared to culturing in CDPF-cultured media, lysosome inhibitor-treated conditioned CDPF media resulted in about 3-folds higher production yield of EVs (FIG. 3B). These results were confirmed by qPCR RQ for fatty acid synthase (FASN) in MSCs cultured in different media including CCM, CCM with lysosome inhibitor, CDPF, and CDPF with lysosome inhibitor (FIG. 3C). Using RT-qPCR, the expression of FASN were about 25 times higher for MSCs cultured in CCM with lysosome inhibitor than that of MSCs cultured in CCM and the expression of FASN were about 5 times higher for MSCs cultured in CDPF with lysosome inhibitor than that of MSCs cultured in CDPF without lysosome inhibitor.

### Example 4: Enriched Production of EVs

This example provides a process of a scalable production of high-quality EVs with high efficiency. Mesenchymal stem cells were seeded on the plate at a density of ˜5000 cells/cm² in the culture medium (17.5% FBS, α-MEM, antibiotics) and the plate was placed in 5% CO₂ humidified incubator. One day after the incubation, the culture media was aspirated to remove non-adherent cells and switched with a fresh media. The plate was incubated again. After further culturing for around 3 to 5 days, the cell was washed three times with PBS, which was pre-warmed at 37° C. water bath, to remove any residual serum from the previous culture medium. The washed cell was cultured in CDPF/lysosome inhibitor medium for 24 to 48 hours. After the designated period of culturing, the culture media was centrifuged at 800×g for 20 minutes to remove cellular debris. The supernatant was directly applied to 0.2 μm filter to remove any aggregates and stored at −80° C. prior to the use.

### Example 5: Anti-Inflammatory Activity Test of EVs

This example demonstrates that in vivo treatment with EVs reduced inflammatory response and the response to EV was dose dependent. Frozen cells (RAW 264.7) were washed in 10 mL previously warmed (37° C.) DMEM supplemented with FBS (10%) and antibiotics (100 U/mL penicillin and 100 mg/mL streptomycin). The cells were centrifuged at 450×g for 10 min, resuspended in 1 mL medium, and cultured in a T75 flask containing 25 mL of the same medium for 24 hours at 37° C. in 5% CO₂. After the cells were detached by gentle scarping with a cell scarper, they were collected, centrifuged (10 min at 450×g), and resuspended in DMEM containing 5% of FBS and antibiotics. The cells were counted and seeded at a density of 1×10⁵ cells per square centimeter using 300 μL medium (5% of FBS plus antibiotics) per well in a 48-well plate. After 20 hours of incubation, the macrophages were stimulated by replacing the 300 mL culturing medium with a similar volume of medium alone (5% FBS and antibiotics) or containing LPS (10 ng/mL), LPS plus dexamethasone (1 mg/mL), or LPS in combination with exosomes (0.5×10⁹ vesicles per milliliter). All the different conditions were prepared in a total volume of 1 mL and tested in triplicates. The conditioned medium was collected after 4 hours of incubation at 37° C. and kept frozen until assayed for cytokines by ELISA.

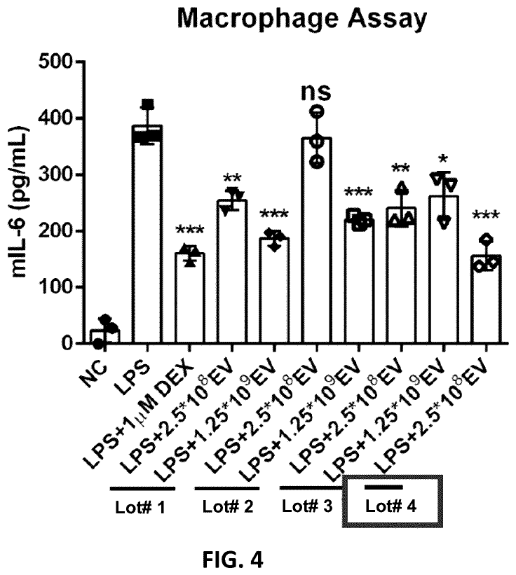

FIG. 4 shows results of anti-inflammatory activity test using 4 different lots of EVs. Macrophages (RAW 264.7) were cultured with one of the EV preparations (Q&R, QE, QF, Chlo) at 2.5×10⁸ EVs/ml or 1.25×10⁹ EVs/ml or a positive control of dexamethasone after inflammation was induced by the addition of LPS. The levels of IL-6, a proinflammatory cytokine, were determined by ELISA. All groups cultured with one of the EVs preparations showed lower IL-6 levels than the positive control, generally in a dose dependent manner.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-26. (canceled)

27. A method for increasing production of extracellular vesicles (EVs), the method comprising:
a. culturing mammalian cells in a chemically-defined, protein-free (CDPF) medium and a lysosome inhibitor to produce a conditioned culture medium, wherein the lysosome inhibitor is chloroquine, wherein a concentration of chloroquine is about 50 μM to about 200 μM;
b. separating the conditioned culture medium after cell culture from the cells; and
c. purifying EVs from the conditioned culture medium.

28. The method of claim 27, wherein the mammalian cells comprise a stem cell, an immune cell, a naïve cell, or an engineered cell.

29. The method of claim 27, wherein the purifying EVs comprises passing the conditioned culture medium over an anion exchange column to isolate EVs having a negative surface charge.

30. The method of claim 27, wherein the EVs have an enriched expression of CD63.

31. The method of claim 30, wherein the enriched expression of CD63 comprises an increase in CD63 expression by the cells cultured in the CDPF medium and the lysosome inhibitor than by cells cultured without the lysosome inhibitor.

32. The method of claim 30, wherein the enriched expression of CD63 comprises an increase of the amount of CD63 expressing exosomes.

33. The method of claim 31, wherein the increase in CD63 expression is about twofold to about ten-fold higher than by cells cultured without the lysosome inhibitor.

34. The method of claim 27, wherein the EVs have a higher anti-inflammatory activity compared to EVs prepared by cells cultured without the lysosome inhibitor.

35. The method of claim 34, wherein the higher anti-inflammatory activity of the EVs comprises at least about 2-fold, 3-fold, 4-fold, 5-fold, 6-fold, or 7-fold higher anti-inflammatory activities, compared to the EVs from cells cultured without the lysosome inhibitor.

36. The method of claim 34, wherein the anti-inflammatory activity of the EVs is measured by IL-6 level in a cell treated with the EVs.

37. The method of claim 27, wherein a level of a proinflammatory biomarker in a cell treated the EVs is lower compared to EVs prepared by cells cultured without the lysosome inhibitor.

38. The method of claim 37, wherein the level of proinflammatory biomarker is lowered by at least 2-fold, 3-fold, 4-fold, 5-fold, 6-fold, or 7-fold as compared to the EVs from cells cultured without the lysosome inhibitor.

39. The method of claim 37, wherein the proinflammatory biomarker comprises IL-6.

40. The method of claim 27, wherein the cells are cultured for between about 24 hours and about 84 hours.

41. The method of claim 27, wherein the culturing the cells in the CDPF medium and the lysosome inhibitor decreases formation of lysosomes and/or autophagosomes by the cells.

42. The method of claim 27, wherein the culturing the cells in the CDPF medium and the lysosome inhibitor upregulates a fatty acid expression by the cells.

43. The method of claim 42, wherein the fatty acid expression comprises fatty acid synthase expression.

44. The method of claim 27, wherein the culturing the cells in the CDPF medium and the lysosome inhibitor reduces internal acidification of the cells.

45. The method of claim 27, wherein the increasing production of EVs comprises an increase in production of EVs.

46. The method of claim 27, wherein the increasing production of EVs comprises a decrease in degradation of EVs.

47. The method of claim 27, wherein the production of EVs is increased by at least about 1.5-fold compared to the production of EVs by cells cultured without the lysosome inhibitor.

48. The method of claim 28, wherein the stem cell comprises a mesenchymal stem cell that is derived from a bone marrow, an umbilical cord, a placenta, or an adipose tissue.

49. (canceled)

50. The method of claim 27, wherein the EVs have with a mean diameter of between about 70 nm and about 280 nm.

51. The method of claim 27, wherein the CDPF medium comprises hypoxanthine, thymidine, glutamine, glucose, essential amino acids, or vitamins, or a combination thereof.

52. The method of claim 27, wherein the cells are cultured in one or more of SP600125, U0126, 3-methyladenine, bafilomycin A-1, LY294002, SB202190, and SB203580.

53. The method of claim 27, wherein the EVs have a negative surface charge.
